(19) United States
(12) Patent Application Publication
(10) Pub. No.: US 2015/0291547 A1
(43) Pub. Date: Oct. 15, 2015

(54) SMALL MOLECULES FOR EXTENDING THE WELL BEING OF CELLS AND METHODS OF USE THEREOF

(71) Applicant: Eng Khuan Seng, Singapore (SG)

(72) Inventor: Eng Khuan Seng, Singapore (SG)

(73) Assignee: NANYANG POLYTECHNIC, SINGAPORE (SG)

(21) Appl. No.: 14/345,846

(22) PCT Filed: Sep. 26, 2011
(86) PCT No.: PCT/SG2011/000332

§ 371 Date: Jun. 16, 2014

(51) Int. Cl. C07D 311/30; C07D 407/04

(52) U.S. Cl. CPC C07D 311/30; C07D 407/04

(57) ABSTRACT

The present invention relates to use of a series of compounds and compositions comprising the same for enhancing telomerase expression and/or activating telomerase activity in a cell or tissue of a subject and for treatment of diseases, disorders and/or conditions capable of being affected by enhanced telomerase expression and/or telomerase activation.

### FIELD OF THE INVENTION

The present invention relates to use of a compound for enhancing telomerase expression and/or activating telomerase activity in cells or tissues.

### BACKGROUND OF THE INVENTION

Human telomerase (hTERT) is an RNA-dependent DNA polymerase that synthesizes chromosomal DNA ends called telomeres, present in humans as repetitive, six base motif (TTAGGG)n of 10-15 kb in length. The major function of telomeres is to protect chromosomes from degradation, end-to-end fusions, rearrangements and chromosome loss; therefore, they are vital for genome stability, gene expression, ageing and cell survival.

In most normal somatic cells (human adult tissues), telomeres becomes shortened upon each cell division (typically 50-200 base pairs of the telomere length are lost) due to the absence of the hTERT enzyme which is required to rebuild the lost telomeres. This progressive shortening event eventually results in irreversible cell growth arrest, termed as cellular senescence (ageing) and eventually apoptosis. In contrast, in highly proliferative cells such as germ cells, stem cells, and in 85-95% of cancers, enhanced telomerase activity can be detected. Hence, the telomerase is involved in the regulation of the proliferative potential of both normal and malignant cells. So far, the expression of hTERT mRNA is found to coincide with telomerase activity pointing to importance of transcriptional control in the regulation of this enzyme.

As mentioned above, high telomerase activity is highly associated in the vast majority of biopsies from all cancer cases studied to date. However, telomerase by itself does not cause cancer. Studies have shown that over-expression of telomerase causes no malignant changes in normal cells, and in developing germ cells (cells that produce oocytes and sperm), telomerase is highly activated yet the cells remain normal. Even though telomerase activation is important for cancer cell survival, telomerase activation in normal human cells is expected to improve their function without causing cancerous changes and thus delay ageing symptoms.

There have been several reports on compounds having the ability to enhance and/or activate telomerase activity in cells and/or tissues and thus, able to treat diseases, disorders and/or conditions related thereto.

U.S. application Ser. No. 12/602,956, publication no. 2010/0267667, entitled “Telomerase activating compounds and methods of use thereof” discloses a series of tri-phenyl compounds and compositions comprising the compounds for activating telomerase and treating diseases, disorders and/or conditions related thereto.

U.S. application Ser. No. 10/563,533, publication no. 2007/0122501, entitled “Formulations containing astragalus extracts and uses thereof” discloses formulations containing plant extracts, in particular Astragalus extracts, and their use in inducing telomerase activity in cells. The formulations are useful for treating diseases subject to treatment by an increase in telomerase activity in selected cells, such as, for example, HIV infection, acute or chronic skin ailments, etc. They are useful for enhancing replicative capacity of cells in culture and are suitable for use as cosmetic formulations for conditioning the skin.

U.S. Pat. No. 7,846,904, entitled “Compositions and methods for increasing telomerase activity” discloses a series of compositions containing compound having at least a glycoside group for increasing telomerase activity in cells.

With the increase in use and demand of these compounds in cosmetics and pharmaceutical products, there is an increasing need to look for a new source of compounds to meet the demand.

Therefore, there is a need to provide compounds that seek to address at least one of the above problems, or at least to provide an alternative.

### SUMMARY OF THE INVENTION

In accordance with a first aspect of the present invention, there is provided a use of a compound for enhancing telomerase expression and/or activating telomerase activity in a cell or tissue and for the treatment of diseases, disorders and/or conditions capable of being affected by enhanced telomerase expression and/or telomerase activation. In particular, the present invention provides a use of a compound represented by the structure of formula (I):

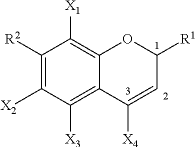

wherein
each of X₁ and X₃ independently represents a hydrogen atom or a hydroxyl group;
   X₂ represents a hydrogen atom or a lower alkoxy group;
   X₄ represents a hydrogen atom or a keto (═O) group together with ═ represents a single bond between carbons 2 and 3 and — represents a double bond between carbons 1 and 2;
   R² represents a hydroxyl group, D-glucopyranoside or a 3-methylbut-2-enyloxy group; and
   R¹ represents a dihydroxyphenyl group, or a keto (═O) group with the proviso that X₄ is not a keto (═O) and at least R¹ or X₄ represents a keto (═O) group; or pharmaceutically acceptable salt, pharmaceutical product or any combination thereof, for the manufacture of a medicament for increasing telomerase expression, activity or a combination thereof in a cell or tissue.

In accordance with a second aspect of the invention, there is provided use of a compound represented by the structure of formula (II) when X₄ represents a keto (═O) group:

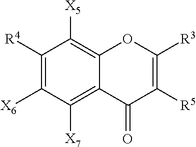

wherein
X₅ represents a hydrogen atom, lower alkenyl group, lower alkoxy group or a hydroxyl group;
X₆ represents a hydrogen atom, halogen atom, lower alkoxy group or an acetoxy group;
X₇ represents a hydrogen atom, lower alkoxy group, a hydroxyl group or an acetoxy group;
R³ represents an aryl group; and
R⁴ represents a hydrogen atom, a hydroxyl group, lower alkoxy group, an acetoxy group or a glycoside;
R⁵ represents a hydrogen atom, an aryl group, a hydroxyl group, a lower alkoxy group or a methylbenzenesulfonate;
   or pharmaceutically acceptable salt, pharmaceutical product or any combination thereof, for the manufacture of a medicament for increasing telomerase expression, activity or a combination thereof in a cell or tissue.

In accordance with some embodiments of the invention, the cell or tissue is isolated from or within a subject having a disease or condition associated with aging, greying of hair or hair loss, a degenerative joint disease, a degenerative disease of musculature, macular degeneration or a degenerative disease of the skeletal system.

In accordance with another aspect of the present invention, there is provided a composition containing said compounds, a pharmaceutically acceptable salt thereof or other derivative thereof as an active ingredient and a pharmaceutical acceptable carrier.

In accordance with a further aspect of the present invention, the present invention includes a method of enhancing and/or activating telomerase activity in a cell or tissue. The method comprising contacting the cell or tissue of a subject with a composition containing an effective amount of a compound of formula (I) or formula (II) or administering to the subject a composition containing an effective amount of a compound of formula (I) or formula (II).

### BRIEF DESCRIPTION OF THE DRAWINGS

The above and other features and advantages of the present invention will more clearly understood from the following detailed description taken in conjunction with the accompanying drawings:

FIG. 1 is a chart showing the percentage change in telomerase expression upon 16 hours incubation with telomerase activating compounds of the present invention;

FIG. 2 is a chart showing the percentage increase in HepG2 cell proliferation upon treatment with telomerase activators.

FIG. 3 is a chart showing a dosage response curve of 7E, P1 (Cynaroside) on hTERT expression.

FIG. 4 is a chart showing a dosage response of 8H, P1 (Capensine) on hTERT expression.

FIG. 5 is a chart showing a dosage response of 11E, P2 (Scopoletin) on hTERT expression.

### DETAILED DESCRIPTION OF THE INVENTION

### I. Definitions

The following words and terms have the meanings given below, unless indicated otherwise. These are intended as general definitions and should in no way limit the scope of the present invention to those terms alone, but are put forth for better understanding of the following description.

The term “pharmaceutically acceptable salts” refers to salts prepared from pharmaceutically acceptable non-toxic bases or acids, including inorganic or organic bases and inorganic or organic acids.

The term “pharmaceutical product” refers, in other embodiment, to a composition suitable for pharmaceutical use (pharmaceutical composition).

The term “effective amount” as used herein is meant an amount sufficient to effect the desired therapeutic benefit. The exact amount will vary with varying conditions such as, but not limited to, age, physical condition of the subject being treated, the specific disease, disorder or condition being treated, the duration of treatment, the severity of the condition being treated, the particular agent being administered, the mode of administration, and so forth. For any given case, an appropriate “effective amount” may be determined by one of ordinary skill in the art using only routine experimentation.

The term “subject” as used herein refers to individuals that can be treated with the present compounds and/or pharmaceutical compositions, also refers to humans and other mammals.

The term “lower alkenyl” as used in definition of X₅ refers, in one embodiment, to a unsaturated aliphatic hydrocarbon, including straight-chain, branched-chain and cyclic alkenyl groups having 1 to 6 carbons, with at least one double bone. Examples of alkenyl groups include but are not limited to vinyl, 1-propenyl, 2-propenyl, isopropenyl, 2-methyl-1-propenyl, 3-methyl-1-propenyl, 2-methyl-2-propenyl, 3-methyl-2-propenyl, 1-butenyl, 2-butenyl, 3-butenyl group, 3-methyl-2-butenyl and the like; preferably a 3-methyl-2-butenyl group.

The term “lower alkoxy group” as used in definitions of X₂, X₅, X₆, X₇, R⁴, R⁵ refers, in one embodiment, to an oxygen atom which is attached to a lower alkyl group, for example, a straight or branched chain alkoxy group having 1 to 6 carbons such as a methoxy, ethoxy, propoxy, isopropoxy, butoxy, isobutoxy, s-butoxy, t-butoxy, pentoxy, isopentoxy, 2-methylbutoxy, 1-ethylpropoxy, 2-ethylpropoxy, neopentoxy, hexyloxy, 4-methylpentoxy, 3-methylpentoxy, 2-methylpentoxy, 3,3-dimethylbutoxy, 2,2-dimethylbutoxy, 2-methylpentoxy, 3,3-dimethylbutoxy, 2,2-dimethylbutoxy, 1,1-dimethylbutoxy, 1,2-dimethylbutoxy, 1,3-dimethylbutoxy or 2,3-dimethylbutoxy group; preferably a C1-C4 alkoxy group; more preferably a C1-C2 alkoxy group; and most preferably a methoxy group.

The term “keto” as used in definitions of X₄, R¹ refers, in one embodiment, to “═O”.

The term an “aryl group” as used in definitions of R³, R⁴ refers, in one embodiment, to a phenyl group; a phenyl group substituted with a methyl group, a methoxy group or a hydroxyl group; or a phenyl group substituted with at least two groups selected from hydroxyl group, methoxy group and acetoxy group.

The term “glycoside” as used in the definition of R⁴ refers to a sugar derivative containing a non-sugar group attached through an oxygen or nitrogen bond. In some embodiments of the invention, the bond is one of oxygen.

The term “halogen atom” as used in definitions of X₆ refers, in one embodiment, to a fluorine, chlorine, bromine or iodine atom; preferably a bromine atom or chlorine atom.

The term “derivative” as used herein refers to a compound that is modified or partially substituted with another component. Additionally, the term “derivative” encompasses compounds that can be structurally similar but can have similar or different functions.

### II. Compounds of the Invention

The present invention relates to use of a series of compounds and compositions comprising the same for stimulating and/or enhancing telomerase expression and/or activating telomerase activity in cells and tissues of a subject and for the treatment of diseases, disorders and/or conditions capable of being affected by enhanced telomerase expression and/or telomerase activation.

In accordance with one embodiment of the invention, conditions capable of being affected by enhanced telomerase expression and/or telomerase activation include ageing of the skin such as dermal atrophy and thinning, elastolysis and skin wrinkling, sebaceous glad hyperplasia or hypoplasia, senile lentigo, pigmentation abnormalities, greying of hair and hair loss or thinning (baldness, alopecia), or chronic skin ulcers.

Diseases and disorders subject to treatment by an increase in telomerase activity include degenerative disease, such as degenerative joint disease, osteoporosis, osteoarthritis, neurodegenerative disease, and other degenerative conditions of the skeletal system, sarcopenia and other degenerative conditions of the musculature; stress-related diseases of the vascular system or age-related macular degeneration which occurs with natural ageing.

The compounds of the present invention can also be used for increasing telomerase expression and/or activity in memory T cells, thereby strengthening immune memory response and response to vaccines or increasing telomerase expression and/or activity in healthy tissue, thus prolonging the lifespan of a subject while sustaining the subject in good health.

In accordance with one embodiment of the invention, the compounds of the present invention are represented by the structure of formula (I):

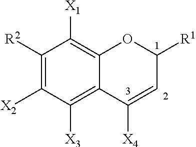

wherein
each of X₁ and X₃ independently represents a hydrogen atom or a hydroxyl group;
   X₂ represents a hydrogen atom or a lower alkoxy group;
   X₄ represents a hydrogen atom or a keto (═O) group together with ═ represents a single bond between carbons 2 and 3 and — represents a double bond between carbons 1 and 2;
   R² represents a hydroxyl group, D-glucopyranoside or a 3-methylbut-2-enyloxy group; and
   R¹ represents a dihydroxyphenyl group, or a keto (═O) group with the proviso that X₄ is not a keto (═O) and at least R¹ or X₄ represents a keto (═O) group.

In selected embodiment of formula (I), each of X₁ and X₂ is a hydrogen atom, X₃ is a hydroxyl group, X₄ is a keto, R¹ is a dihydroxyphenyl and R² is a D-glycopyranoside. This compound is 2-(3,4-dihydroxyphenyl)-5-hydroxy-7-[3,4,5-trihydroxy-6-(hydroxymethyl)(2H-3,4,5,6-tetrahydropyran-2-yloxy)]chromen-4-one and is also known as Cynaroside. This compound has the structure of formula (III):

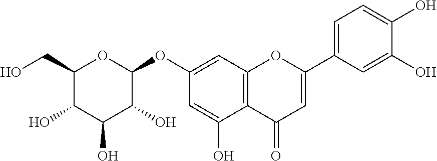 [SMILES: O=c1cc(-c2ccc(O)c(O)c2)oc2cc(O[C@@H]3O[C@H](CO)[C@@H](O)[C@H](O)[C@H]3O)cc(O)c12]

In another selected embodiment of formula (I), each of X₃ and X₄ is a hydrogen atom, X₁ is a hydroxyl group, X₂ is a methoxy group, R¹ is a keto group and R² is a 3-methylbut-2-enyloxy group. This compound is 8-hydroxy-6-methoxy-7-(3-methylbut-2-enyloxy) chromen-2-one and is also known as Capensine. This compound has the structure of formula (IV):

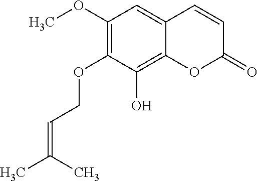 [SMILES: COc1cc2ccc(=O)oc2c(O)c1OCC=C(C)C]

In further selected embodiment of formula (I), each of X₁, X₃ and X₄ is a hydrogen atom, X₂ is a methoxy group, R¹ is a keto group and R² is a hydroxyl group. This compound is 7-hydroxy-6-methoxychromen-2-one and is also known as Scopoletin. This compound has a structure of formula (V):

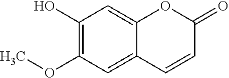

Other compounds having the backbone structure of 2-(3,4-dihydroxyphenyl)-5-hydroxy-7-[3,4,5-trihydroxy-6-(hydroxymethyl)(2H-3,4,5,6-tetrahydropyran-2-yloxy)]chromen-4-one are also considered for use in the present invention. Some of these compounds are listed in Table 2 below. These compounds generally are represented by the structure of formula (II):

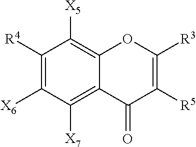

wherein
X₅ represents a hydrogen atom, lower alkenyl group, lower alkoxy group or a hydroxyl group;
X₆ represents a hydrogen atom, halogen atom, lower alkoxy group or an acetoxy group;
X₇ represents a hydrogen atom, lower alkoxy group, a hydroxyl group or an acetoxy group;
R³ represents an aryl group;
R⁴ represents a hydrogen atom, a hydroxyl group, lower alkoxy group, an acetoxy group or a glycoside; and
R⁵ represents a hydrogen atom, an aryl group, a hydroxyl group, lower alkoxy group or a methylbenzenesulfonate.

In accordance with another embodiment of the present invention, the invention provides a composition comprising the compound of the present invention or use of the compound of this invention, for increasing telomerase activity and/or expression in a cell or tissue; and/or treating a condition by increasing telomerase activity and/or expression in cells or tissue of a subject; and/or treating diseases, disorders and/or conditions associated with ageing of a human body or skin.

In general, suitable pharmaceutical composition may be prepared according to methods which are known to those of ordinary skill in the art. The composition comprising the active compounds disclosed herein and may include a conventional pharmaceutical carriers or diluent, and in addition, may include other medicinal agents, pharmaceutical agents, carriers, adjuvants and the like.

In accordance with a further embodiment of the present invention, the invention includes a method of enhancing and/or activating telomerase activity in a cell or tissue. The method comprising contacting the cell or tissue of a subject with a composition containing an effective amount of a compound of formula (I) or formula (II) or administering to the subject a composition containing an effective amount of a compound of formula (I) or formula (II).

Preferably, the method comprises contacting the cell or tissue of a subject with a composition containing an effective amount of a compound selected from the group consisting of 2-(3,4-dihydroxyphenyl)-5-hydroxy-7-[3,4,5-trihydroxy-6-(hydroxymethyl)(2H-3,4,5,6-tetrahydropyran-2-yloxy)]chromen-4-one, 8-hydroxy-6-methoxy-7-(3-methylbut-2-enyloxy) chromen-2-one and 7-hydroxy-6-methoxychromen-2-one.

### III. Sources and Syntheses of Compounds of the Invention

The compounds of the present invention having telomerase activating properties were identified through a low throughput screening of 160 compounds from the TimTec Natural product library (NPL)*. NPL which consists of a total of 480 different pure natural product compounds. The compounds were identified using a stable HepG2 human telomerase promoter construct assay (HepG2-phTERT-SEAP) similar to the ones reported by Huang et. al, (2003) “Activation of Human Telomerase Reverse Transcriptase Expression by Some New Symmetrical Bis-Substituted Derivatives of the Anthraquinone”, J. Med. Chem. 46, 3300-3307.

From the 160 compounds screened using the HepG2-phTERT-SEAP stable cell line, 3 potential telomerase activators were identified. They were from wells 7E and 8H, Plate 1(P1) and 11E, Plate 2 (P2).

Well 7E, P1 contains Cynaroside, well 8H, P1 contains Capensine and well 11E, P2 contains Scopoletin.

The compounds of the present invention alternatively can also be isolated or synthesized from naturally occurring material. For example, the compound, Luteolin 7-O-D-glucopyranoside, can be isolated from dandelion coffee, in Ferula varia and F. foetida, in Campanula persicifolia and C. rotundifolia, in bamboo of species Phyllostachys nigra and in Cynara scolymus (antichoke).

The compound, Capensin, can be isolated from the roots of Bupleurum fruticosum or Haplophyllum obtusifolium.

Scopoletin can be isolated from the roots of plants in the genus Scopolia, such as Scopolia camiolica or Scopolia japonica.

### IV. Determination of Biological Activity

### A. Assay Protocol

“SEAP” stands for secreted alkaline phosphatase enzyme. SEAP was used as a reporter system to monitor the transcriptional activity of hTERT. Here, about 10,000 cells per well were grown in 96-well plates and incubated at 37° C. for 22 hours. Varying types and amounts of natural compounds were added and cells were incubated for another 16 hours. Culture media were collected and heated at 65° C. for 10 mins to inactivate heat labile phosphatases. An equal amount of SEAP buffer (2M diethanolamine, 1 mM MgCl2, and 20 mM L-homoarginine), was added to the media and p-nitrophenyl phosphate was added to a final concentration of 12 mM. Absorbance at 405 nm was taken, and the percentage change in absorbance was calculated.

An increase in SEAP activity detected via the utilization of a substrate, translates to an increase in hTERT expression.

Cell lines suitable for use in the assay were prepared from ATTC No. HB-8065 Hep G2 (Hepatocellular carcinoma, human). The Hep G2 cell line was isolated from a liver biopsy of a 15-year-old male Caucasian with a well differentiated hepatocellular carcinoma. These cells are epithelial in morphology. These cells secrete a variety of major plasma proteins e.g., alpha-fetoprotein (alpha fetoprotein); albumin; alpha2 macroglobulin (alpha-2-macroglobulin); alpha1 antitrypsin (alpha-1-antitrypsin); transferrin; alpha1 antichymotrypsin; (alpha-1-antichymotrypsin); haptoglobin; ceruloplasmin; plasminogen; complement (C4); C3 activator; fibrinogen; alpha1 acid glycoprotein (alpha-1 acid glycoprotein); alpha2 HS glycoprotein (alpha-2-HS-glycoprotein); beta lipoprotein (beta-lipoprotein); retinol binding protein (retinol-binding protein). The cells are also responsive to stimulation with human growth hormone. HepG2 cells and its derivatives are commonly used as a model system for studies of liver metabolism and toxicity of xenobiotics, the detection of cytoprotective, anti (environmental and dietary) genotoxic and cogenotoxic agents, understanding hepatocarcinogenesis, and for drug targeting studies. HepG2 cells are also employed in trials with bio-artificial liver devices.

The plasmids used in the test were obtained from the Institute of Biopharmaceutical Science, National Yang-Ming University, Shih-Pai, Taipei 112, Taiwan, Republic of China.

One of the plasmids contains the 5′ upstream hTERT promoter region followed by the reporter gene, the SEAP gene, while the other plasmid contains the CMV promoter region controlling the expression of the SEAP gene.

Each plasmid was then individually transfected into HepG2 cells and stable cells lines were established by selection on G418 (1 mg/ml). Stable clones of from each plasmid transfected cell line was established and each clone was propagated and preserved under liquid nitrogen for long term storage prior to use

The newly established HepG2-pTERT-SEAP and HepG2-pCMV-SEAP stable cell lines were then used for screening for inhibitors or activators of human telomerase expression.

An increase in SEAP activity indicates an increase in hTERT expression and a decrease in SEAP activity indicates a decrease in hTERT expression.

HepG2-pCMV-SEAP serves as a positive control to ensure that SEAP is always secreted by cells harbouring this plasmid. It also serves as a specificity control as any small molecules that stimulate or inhibit only telomerase expression identified using the HepG2-pTERT-SEAP cells screening should show little to no increase in SEAP activity.

### B. Exemplar Assay Results

HepG2-phTERT-SEAP cells were grown for 22 hours in M199 medium supplemented with 10% FBS. The three compounds, 7E (P1), 8H(P1) and 11E(P2) (20 μg/ml) specified above were added and incubated for another 16 hours. After 16 hours, SEAP assay was conducted in accordance to the protocol described by Huang et al. (2003). Results were as shown in FIG. 1 which shows the average increase in hTERT expression from n=5 independent experiments.

As shown in FIG. 1, incubation of compounds 7E (P1), 8H(P1) and 11E(P2) at 20 μg/ml shows an average increase in hTERT expression of 29.42%, 49.76% and 57.99% respectively after 16 hours of incubation in the HepG2-hTERT reporter cell line as indicated by the increase in SEAP activity.

To check the specificity of the three compounds 7E (P1), 8H(P1) and 11E(P2) in activating the expression of hTERT, an experiment as described below was conducted.

HepG2-phTERT-SEAP cells were grown for 22 hours in M199 medium supplemented with 10% FBS. The three compounds (20 μg/ml) specified above were then added and incubated for another 16 hours. After 16 hours, SEAP assay was conducted in accordance to the protocol described by Huang et al. (2003). Concurrently, HepG2-pCMV-SEAP cells were also subjected to the compounds and results were also collected. The results obtained were as shown in Table 1 below which indicate the change in hTERT expression from n=3 independent experiments.

TABLE 1
Ratio of activity of hTERT promoter relative to CMV promoter
driven SEAP plasmid in HepG2 cells upon compound treatment

| Compounds | Average phTERT/pCMV ratio* |
|---|---|
| 7E, Plate 1 | 1.08 ± 0.04 |
| 8H, Plate 1 | 2.16 ± 0.14 |
| 11E, Plate 2 | 2.01 ± 0.01 |
| Suramin (Positive Control) | 1.16 ± 0.11 |

*Average values including standard error (SE) values were calculated from results from n = 3 independent experiments conducted.

Average values of phTERT/PCMV ratio above indicate that the compound(s) are able to specifically stimulate hTERT expression. As shown in Table 1, compounds 8H, 11 E and 7E displayed the highest to lowest specificity in activating hTERT expression. Compounds 8H and 11E, is highly specific in activating hTERT expression (average phTERT/pCMV ratio of more than 2) while 7E is only mildly specific (average phTERT/pCMV ratio of just above 1). This is in accordance to Bao et al. (2002), “Activation of cancer-specific gene expression by surviving promoter”, J. Natl. Cancer Inst., 94, 522-528; results which reported that, a ratio of 2 is highly suggestive of the specificity of the activation of the promoter under investigation using the same SEAP reporter system. Hence, compounds 8H and 11E qualify as highly specific in activating telomerase expression while compound 7E only marginally activates hTERT expression.

Additionally, studies have also shown that the presence of CMV promoter in expression analysis will always result in a higher expression of SEAP as compared to levels achieved using hTERT promoter. A similar observation was also made in non-compound treated HepG2-pCMV-SEAP cells, where a higher expression was also observed as compared to HepG2-phTERT-SEAP cells. The same observation corroborated with Murofushi et al. (2006), “Cell cycle-specific changes in hTERT promoter activity in normal and cancerous cells in adenoviral gene therapy: A promising implication of telomerase-dependent targeted cancer gene therapy”, Intl. J. Oncology, 29, 681-688, findings in their LacZ-promoter construct studies with pCMV and phTERT in HepG2 cells. Another study by Kuppuswamy et al. (2005), “Oncolytic adenovirus that overproduces ADP and replicates selectively in tumors due to hTERT promoter-regulated E4 gene expression”, Gene Therapy, 12, 1608-1617, which uses Luc-promoter construct studies with pCMV and phTERT in Hep3B cells (another type of hepatocellular carcinoma cells) also demonstrated similar results. Hence, the significance of the identified compounds, 8H, 11E and 7E in activating telomerase expression is likely to be much higher than basal level expression and also exhibits greater specificity.

### C. Exemplar Cell Proliferation Results

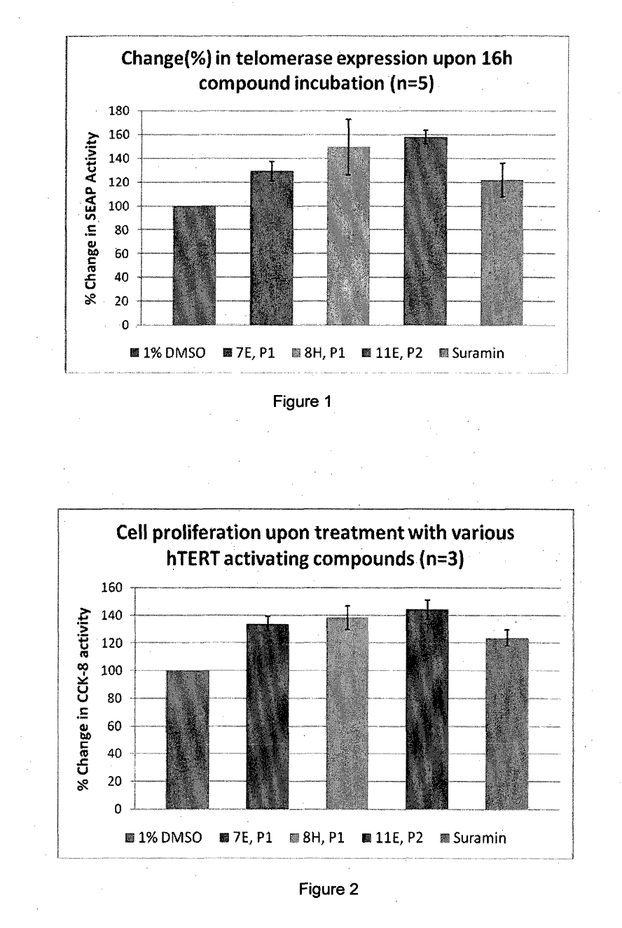

FIG. 2 shows the percentage increase in HepG2 cell proliferation upon treatment with telomerase activators.

HepG2-phTERT-SEAP cells were incubated with hTERT activator compounds at 20 μg/ml and after 16 hours, cell proliferation was assessed using the CCK-8 assay (Dojindo Laboratories). Results shown in FIG. 2 indicate an average increase in cell proliferation from n=3 independent experiments.

From FIG. 2, one can see that the incubation of hTERT activator compounds to HepG2-phTERT-SEAP cells for 16 hours resulted in an average increase in cell proliferation by 34%, 38% and 44% respectively as compared to control wells (1% DMSO) which are not treated with the compounds.

Collectively, the results from FIGS. 1 and 2 indicate that an increase in telomerase activity (expression) generally correlates with an increase in growth rate (increase in cell proliferation). These findings are in line with the findings reported in Holt et al. (1997), “Lack of cell cycle regulation of telomerase activity in human cells”, Proc. Natl. Acad. Sci. 94, 10687-10692.

### D. Exemplar Dose Response Results

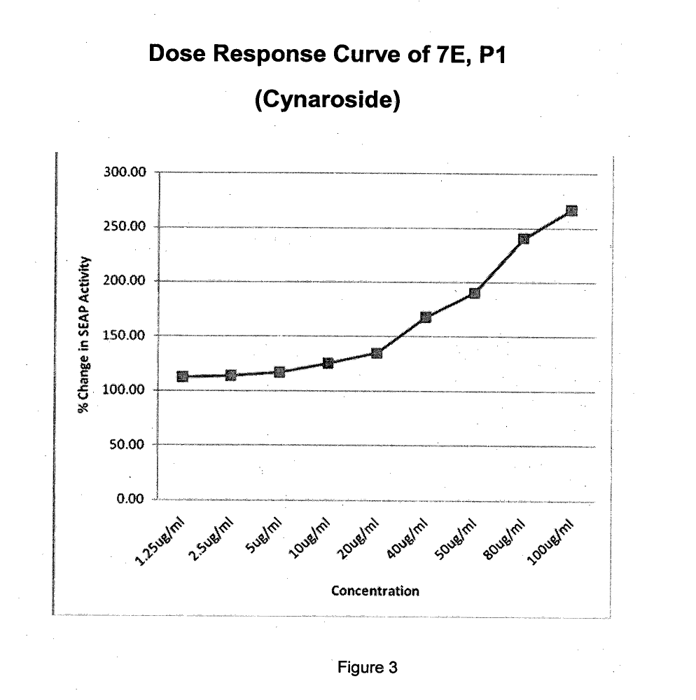

FIG. 3 shows a dosage response curve of 7E, P1 (Cynaroside) on hTERT expression.

From FIG. 3, one can see that the incubation of compounds to HepG2-phTERT-SEAP cells for 16 hours resulted in a dose dependent increase in hTERT expression. At the lowest dosage of 1.25ug/ml, compound 7E, Cynaroside increased hTERT expression by 12.3% above cells which did not receive Cynaroside treatment. At 20 ug/ml and 100 ug/ml, Cynaroside stimulated an increase in hTERT expression by 34.7% and 167.0% when compared to control wells receiving 1% DMSO only.

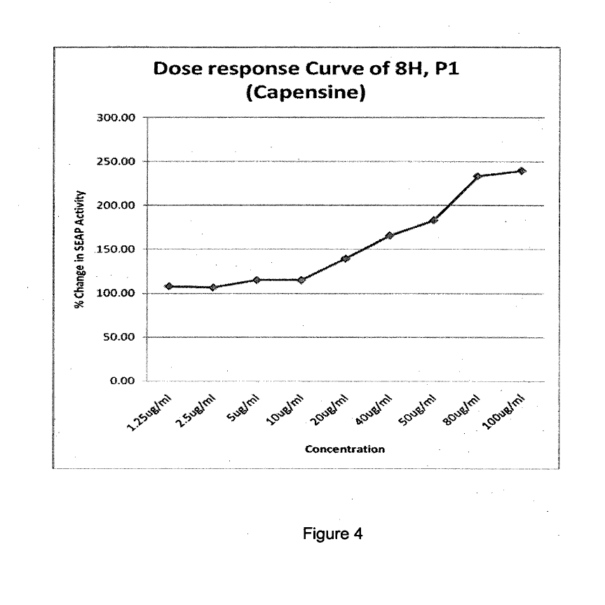

FIG. 4 shows a dosage response curve of 8H, P1 (Capensine) on hTERT expression.

From FIG. 4, one can see that the incubation of the compounds to HepG2-phTERT-SEAP cells for 16 hours resulted in a dose dependent increase in hTERT expression. At the lowest dosage of 1.25ug/ml of compound 8H, Capensine increased hTERT expression by 8.1% above cells which did not receive Capensine treatment. At 20 ug/ml and 100 ug/ml, capensine stimulated an increase in hTERT expression by 39.5% and 139.5% when compared to control wells receiving 1% DMSO only.

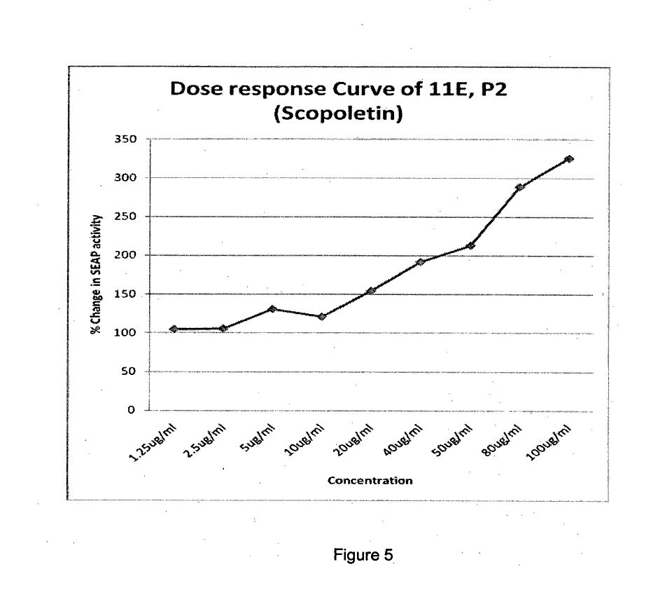

FIG. 5 shows a dosage response curve of 11E, P2 (Scopoletin) on hTERT expression.

From FIG. 5, one can see that the incubation of the compounds to HepG2-phTERT-SEAP cells for 16 hours resulted in a dose dependent increase in hTERT expression. At the lowest dosage of 1.25ug/ml of compound 8H, Scopoletin increased hTERT expression by 4.3% above cells which did not receive Scopoletin treatment. At 20 ug/ml and 100 ug/ml, Scopoletin stimulated an increase in hTERT expression by 54.2% and 225.8% when compared to control wells receiving 1% DMSO only.

### V. Selection of Additional Compounds

Additional compounds effective to increase telomerase activity were also selected by screening derivatives of compounds of formula (II) in a SEAP assay, using the same method used for screening the above three compounds, 7E, P1; 8H, P1; and 11E, P2.

The exemplary compounds listed in Table 2 below are derivatives of Cynaroside selected from the screening, which are potential telomerase activators.

TABLE 2

| Potential* & | | |
|---|---|---|
| Probable hTERT | | Average |
| Activators | | hTERT |
| (Plate) Well | | expression |
| ID Number | Chemical Structures | (%) |
| ^{*}(Plate 6) G7 ST083655 | 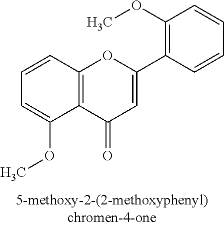 [SMILES: COc1ccccc1-c1cc(=O)c2c(OC)cccc2o1] | 267.85 |
| (P4) B9 ST069306 | 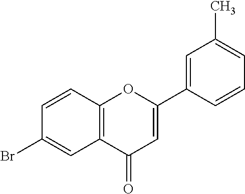 [SMILES: Cc1cccc(-c2cc(=O)c3cc(Br)ccc3o2)c1] | 220.73 |
| ^{*}(Plate 6) D10 ST086260 | 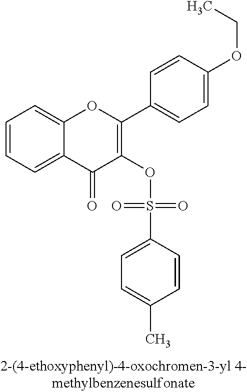 [SMILES: CCOc1ccc(-c2oc3ccccc3c(=O)c2OS(=O)(=O)c2ccc(C)cc2)cc1] | 218.37 |
| (P7) G2 ST092297 | 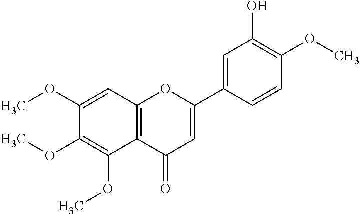 | 210.00 |
| (P7) C4 ST081960 | 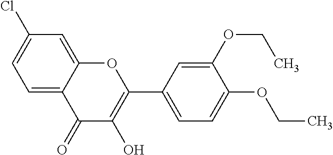 [SMILES: CCOc1ccc(-c2oc3cc(Cl)ccc3c(=O)c2O)cc1OCC] | 196.13 |
| (P2) D8 ST055367 | 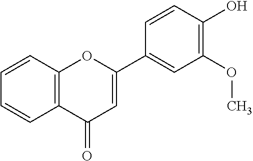 [SMILES: COc1cc(-c2cc(=O)c3ccccc3o2)ccc1O] | 180.35 |
| (P4) A4 ST060287 | 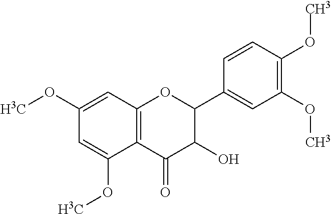 [SMILES: COc1ccc(C2Oc3cc(O[3CH3])cc(O[3CH3])c3C(=O)C2O)cc1OC] | 178.35 |
| (P3) B3 ST056204 | 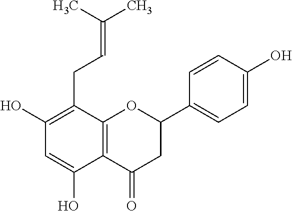 [SMILES: CC(C)=CCc1c(O)cc(O)c2c1OC(c1ccc(O)cc1)CC2=O] | 159.01 |
| ^{*}(Plate 6) E10 ST086510 | 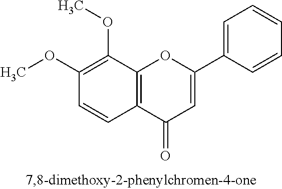 [SMILES: COc1ccc2c(=O)cc(-c3ccccc3)oc2c1OC] | 154.90 |
| (P2) G9 ST055983 | 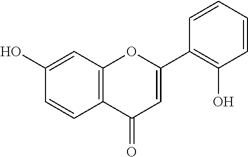 [SMILES: O=c1cc(-c2ccccc2O)oc2cc(O)ccc12] | 149.44 |
| (P3) F2 ST056012 | 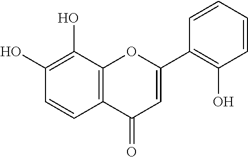 [SMILES: O=c1cc(-c2ccccc2O)oc2c(O)c(O)ccc12] | 147.86 |
| ^{*}(P1) E9 ST024703 | 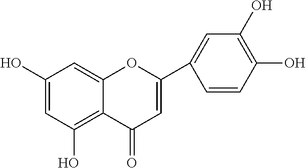 [SMILES: O=c1cc(-c2ccc(O)c(O)c2)oc2cc(O)cc(O)c12] | 146.83 |
| (P2) F9 ST055982 | 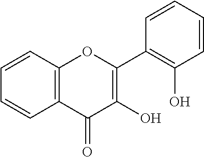 [SMILES: O=c1c(O)c(-c2ccccc2O)oc2ccccc12] | 145.00 |
| *(Plate 6) H4 ST081389 | 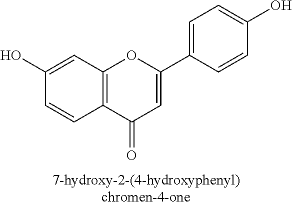 [SMILES: O=c1cc(-c2ccc(O)cc2)oc2cc(O)ccc12] | 144.78 |
| ^{*}(P1) F9 ST024704 | 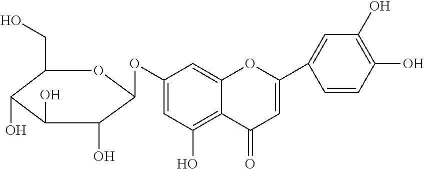 | 144.75 |
| ^{*}(P1) E10 ST024734 | 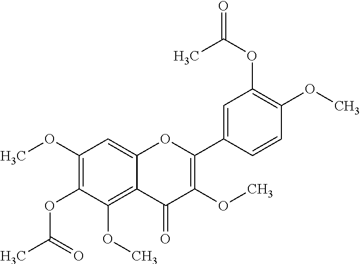 | 141.61 |
| (P2) F10 ST055991 | 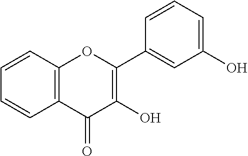 [SMILES: O=c1c(O)c(-c2cccc(O)c2)oc2ccccc12] | 138.53 |
| (P3) H2 ST056014 | 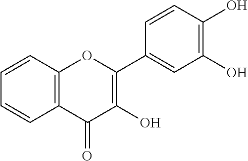 [SMILES: O=c1c(O)c(-c2ccc(O)c(O)c2)oc2ccccc12] | 135.97 |
| (P1) F7 ST023308 | 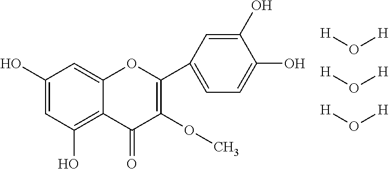 | 133.91 |
| (P1) B9 ST024699 | 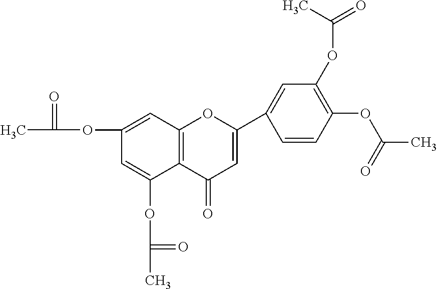 [SMILES: CC(=O)Oc1cc(OC(C)=O)c2c(=O)cc(-c3ccc(OC(C)=O)c(OC(C)=O)c3)oc2c1] | 132.59 |
| (P2) F3 ST038325 | 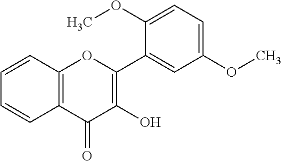 [SMILES: COc1ccc(OC)c(-c2oc3ccccc3c(=O)c2O)c1] | 131.60 |
| (P7) E3 ST008330 | 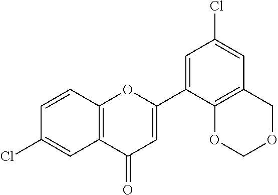 [SMILES: O=c1cc(-c2cc(Cl)cc3c2OCOC3)oc2ccc(Cl)cc12] | 131.43 |
| (P4) D2 ST059620 | 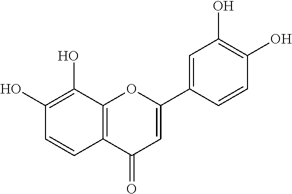 [SMILES: O=c1cc(-c2ccc(O)c(O)c2)oc2c(O)c(O)ccc12] | 131.25 |
| (P3) C9 ST057649 | 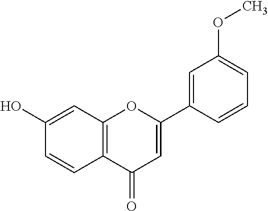 [SMILES: COc1cccc(-c2cc(=O)c3ccc(O)cc3o2)c1] | 129.86 |
| (P3) B5 ST0569259 | 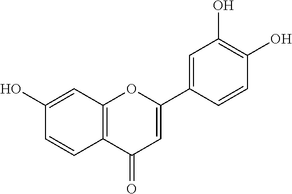 [SMILES: O=c1cc(-c2ccc(O)c(O)c2)oc2cc(O)ccc12] | 129.53 |
| (Plate 6) A8 ST083657 | 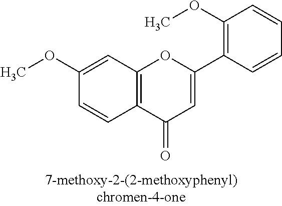 [SMILES: COc1ccc2c(=O)cc(-c3ccccc3OC)oc2c1] | 126.62 |
| (P3) H4 ST056257 | 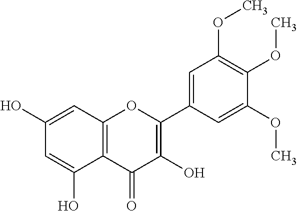 [SMILES: COc1cc(-c2oc3cc(O)cc(O)c3c(=O)c2O)cc(OC)c1OC] | 124.78 |

The above is a description of the subject matter the inventor regards as the invention. It is foreseeable that those skilled in the art can and will design alternative methods and compounds that include this invention based on the above disclosure.

1. A method for increasing telomerase expression, activity or a combination thereof in a cell or tissue, comprising administration of a compound, or a pharmaceutically acceptable salt, pharmaceutical product or any combination thereof, wherein the compound is represented by the structure of formula (I): 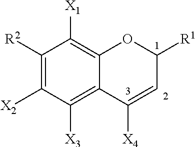
wherein
each of X₁ and X₃ independently represents a hydrogen atom or a hydroxyl group;
X₂ represents a hydrogen atom or a lower alkoxy group;
X₄ represents a hydrogen atom or a keto (═O) group;
R² represents a hydroxyl group, D-glucopgranoside or a 3-methylbut-2-enyloxy group; and
R¹ represents a dihydroxyphenyl group, or a keto (═O) group,
with the proviso that X₄ is not a keto (═O) and at least R¹ or X₄ represents a keto (═O) group.

2. The method of claim 1, wherein each of and X₂ is a hydrogen atom, X₃ is a hydroxyl group, X₄ is a keto, R¹ is a dihydroxyphenyl and R² is a D-glycopyranoside.

3. The method of claim 1, wherein each of X₃ and X₄ is a hydrogen atom, X₁ is a hydroxyl group, X₂ is a methoxy group, R¹ is a keto group and R² is a 3-methylbut-2-enyloxy group.

4. The method of claim 1, wherein each of X₁, X₃ and X₄ is a hydrogen atom, X₂ is a methoxy group, R¹ is a keto group and R² is a hydroxyl group.

5. The method of claim 2, wherein the compound is 2-(3,4-dihydroxyphenyl)-5-hydroxy-7-[3,4,5-trihydroxy-6-(hydroxymethyl)(2H-3,4,5,6tetrahydropyran-2-yloxy)]chromen-4-one.

6. The method of claim 3, wherein the compound is 8-hydroxy-6-methoxy-7-(3-methylbut-2-enyloxy) chromen-2-one.

7. The method of claim 4, wherein the compound is 7-hydroxy-6-methoxychromen-2-one.

8. The method of claim 1, wherein when X₄ is a keto (═O) group, the compound is represented by the structure of formula (II): 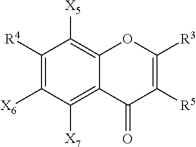
wherein
X₅ represents a hydrogen atom, lower alkenyl group, lower alkoxy group or a hydroxyl group;
X₆ represents a hydrogen atom, halogen atom, lower alkoxy group or an acetoxy group;
X₇ represents a hydrogen atom, lower alkoxy group, alkoxy group, a hydroxyl group or an acetoxy group;
R³ represents an aryl group;
R⁴ represents a hydrogen atom, a hydroxyl group, lower alkoxy group, an acetoxy group or a glycoside; and
R⁵ represents a hydrogen atom, an aryl group, a hydroxyl group, a lower alkoxy group or a methylbenzenesulfonate.

9. The method of claim 1, wherein the cell or tissue is isolated from or within a subject having a disease or condition associated with aging, greying of hair or hair loss, a degenerative joint disease, a degenerative disease of musculature, macular degeneration or a degenerative disease of the skeletal system.

10. A composition comprising a compound of claim 1 or a pharmaceutically acceptable salt thereof; and a pharmaceutically acceptable carrier.
